(19) United States
(12) Patent Application Publication
(10) Pub. No.: US 2011/0118741 A1
(43) Pub. Date: May 19, 2011

(54) APPARATUS FOR PREVENTING DISTAL MIGRATION OF LAG SCREW FOR HIP FRACTURE SURGERY

(76) Inventors: Jin Gon Kim, Gyeongsangbuk-do (KR); Myung Rae Cho, Gyeongsangbuk-do (KR)

(21) Appl. No.: 11/914,218

(22) PCT Filed: May 10, 2006
(86) PCT No.: PCT/KR06/01747

§ 371 Date: May 13, 2008

(30) Foreign Application Priority Data
May 12, 2005 (KR) 20-2005-0013366

(51) Int. Cl. A61B 17/58

(52) U.S. Cl. 606/65

(57) ABSTRACT

A device for preventing distal migration of a LAG screw used for hip joint fracture surgery is disclosed. A locking support (10) has a Side Plate and Nail Rod (11) and a LAG Screw Insertion Opening (13) which are bent relative to each other. The Side Plate and Nail Rod (11) has a plurality of Plate and nail screw hole (12). The LAG Screw Insertion Opening (13) has a LAG Screw Sliding Hole (14). A Depressed Stop Groove (15) is defined on one side or on each of opposite sides on the inner surface of the LAG Screw Insertion Opening (13). A Shank of LAG screw (20) to be inserted into the LAG Screw Sliding Hole (14) has a Screw Thread (21) which is formed on the proximal end thereof and a Sliding Stop Apparatus (Elevated Knot) (22) which is formed on one side or on each of opposite sides on the outer surface of the Shank of LAG screw (20) to prevent migration of the distal end of the Shank of LAG screw (20).

### TECHNICAL FIELD

The present invention relates to a device for preventing distal migration of a Lag screw used for hip joint fracture surgery, and more particularly to a migration prevention device which can solve problems occurring due to excessive migration of the distal end of a Shank of LAG screw used for repositioning of the fractured part of a hip joint, such as malunion and nonunion of the fractured part, thereby preventing complications from arising.

### BACKGROUND ART

As is generally known in the art, osteoporosis is a bone disease in which the amount of inorganic constituents and organic constituents of bones abruptly and excessively decrease at substantially the same rate even though the bones have no configurational abnormality. When osteoporosis occurs, holes such as those in coarse pumice or a sponge grow larger and more numerous, thus the bones become increasingly porous, brittle, and subject to fracture. In osteoporosis, the size and volume of the bone do not change, but the mass of the bone is lost at an accelerated rate. While osteoporosis may develop as a result of hormone-dependent diseases or other diseases, it is usually common in older persons.

While aging of bones occurs in every person, when it occurs faster and more seriously for a person's age, it is determined that osteoporosis has developed. In this case, since the bone has an increased risk of fracture even when receiving small shocks, more attention must be paid not only for treatment but also for prevention of further development of the disease.

A small amount of bony tissue loss inevitably occurs in women after 45 and in men after 50 or 60. Osteoporosis occurs when a considerable amount of bony tissue loss is induced such that clinical symptoms result. Osteoporosis develops most frequently in women after menopause, and older men and women lose bone mass at about the same rate, which is called senile osteoporosis.

The total amount of skeletal material is lost from cortical bone and trabecular bone. In cortical bone, the bone tissue is significantly reduced along the surface of an endosteum. While the full body super skeleton is usually afflicted, in some cases, the bony tissue loss is concentrated in the bones of the arms and the legs. If bony tissue loss occurs in the vertebrae, the femoral neck, the pelvis, and the ribs, severe clinical symptoms result. As the weakness of the skeletal system is increased, fine fractures occur, which are then accompanied by pain, skeletal deformation and large fractures.

When osteoporosis occurs in the arms and the legs, if a person falls, the femoral portion is likely to be fractured and the person cannot walk. In the case of women, the frequency of hip joint fracture increases 4 times every 10 years after 50 and 20% of such patients die. For reference, the term “hip joint” indicates the joint between the femur and the acetabulum of the pelvis.

The dynamic compression hip screw and proximal femoral nailing which are currently used in the treatment of pertrochanteric fracture due to osteoporosis occurring in aged persons, serve as a fracture repositioning device for surgery. Referring to FIG. 1, this device comprises a LAG screw composed of Shank of LAG screw 20 and Screw Thread 21, and side plate composed of LAG Screw Sliding Hole 14 with LAG Screw Insertion Opening 13 on one part and Side Plate and Nail Rod 11 with Plate and nail screw hole 12 on the other part.

However, when repositioning a fractured part using the device constructed as mentioned above, problems such as malunion and nonunion of the fractured part occur due to excessive migration of the distal end of the Shank of LAG screw, thus adversely influencing the treatment of the patient.

### DISCLOSURE

### Technical Problem

Accordingly, the present invention has been made keeping in mind the above problems occurring in the prior art, and an object of the present invention is to provide a bone migration prevention device for surgery which can solve the problems occurring due to excessive migration of the distal end of a Shank of LAG screw used for repositioning of the fractured part of a hip joint, such as malunion and nonunion of the fractured part, thereby preventing complications from arising.

### Technical Solution

In order to achieve the above object, according to one aspect of the present invention, there is provided a device for preventing distal migration of a LAG screw used for hip joint fracture surgery, comprising a locking support 10 having a Side Plate and Nail Rod 11 on one part thereof and a LAG Screw Insertion Opening composed of LAG Screw Sliding Hole 14 having a the LAG Screw Insertion Opening 13 on the other part thereof, which are bent relative to each other, the Side Plate and Nail Rod 11 having a plurality of Plate and nail screw hole 12 through which the Side Plate and Nail Rod 11 is locked to a bone, LAG Screw Sliding Hole 14 having a the LAG Screw Insertion Opening 13 and a Depressed Stop Groove 15 being defined on one side or on each of opposite sides in the middle of the inner surface of LAG Screw Insertion Opening 13 which defines the LAG Screw Sliding Hole 14; and a Shank of LAG Screw 20 inserted into LAG Screw Sliding Hole 14 through the LAG Screw Insertion Opening 13, and a Screw Thread 21 which is formed on the proximal end of LAG screw thereof, and a Sliding Stop Apparatus(Elevated knot) 22 which is formed on one side or on each of opposite sides in the middle of the outer surface of the Shank of the LAG screw 20 to prevent migration of the distal end of the LAG screw.

In order to achieve the above object, according to another aspect of the present invention, there is provided a device for preventing distal migration of a LAG screw used for hip joint fracture surgery, comprising a LAG screw 20 having a Screw Thread 21 which is formed on a proximal end thereof and a Depressed Sliding Groove 15a which is defined on one side or on each of opposite sides in the middle of the outer surface of the Shank of LAG screw 20; and a locking support 10 having a Side Plate and Nail Rod 11 on one part thereof and a LAG Screw Insertion Opening composed of LAG Screw Sliding Hole 14 having a LAG Screw Insertion Opening 13 on the other part thereof which are bent relative to each other, Side Plate and Nail Rod 11 having a plurality of Plate and nail screw hole 12 through which the Side Plate and Nail Rod 11 is locked to a bone, a Sliding Stop Apparatus (Elevated Stop Ridge) 22a being projectedly formed on one side or on each of opposite sides in the middle of the inner surface of the LAG Screw Sliding Hole 14 to prevent migration of the distal end of the LAG screw and engaged with Depressed Sliding Groove 15a.

### DESCRIPTION OF DRAWINGS

FIG. 1 is a perspective view illustrating a conventional device used for fracture repositioning surgery;

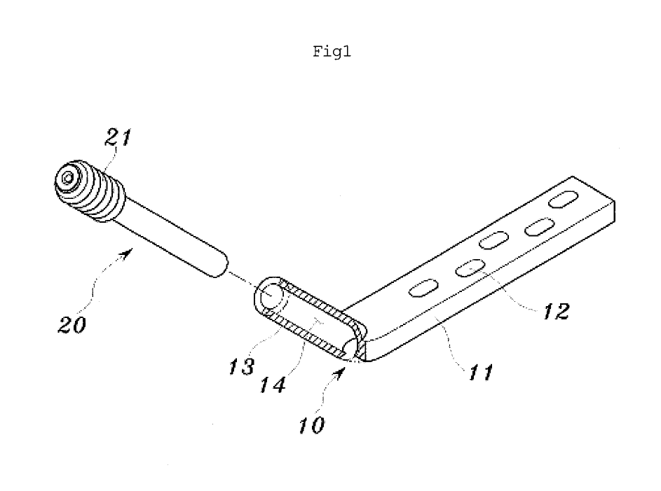

FIG. 2 is a perspective view illustrating a device for preventing distal migration of a LAG screw used for hip joint fracture surgery in accordance with a first embodiment of the present invention;

FIG. 3 is a perspective view illustrating a device for preventing distal migration of a LAG screw used for hip joint fracture surgery in accordance with a second embodiment of the present invention;

FIG. 4 is a schematic view illustrating the use of the device for preventing distal migration of a LAG screw used for hip joint fracture surgery in accordance with the first embodiment of the present invention; and

FIG. 5 is a schematic view illustrating the use of the device for preventing distal migration of a LAG screw used for hip joint fracture surgery in accordance with the second embodiment of the present invention.

### DESCRIPTION OF REFERENCE NUMERALS FOR MAIN PARTS OF DRAWINGS

10: locking support 11: Side Plate and Nail Rod

12: Plate and nail screw hole

13: LAG Screw Insertion Opening

14: LAG Screw Sliding Hole

15: Depressed Stop Groove

15a: Depressed Sliding Groove

20: Shank of LAG screw 21: Screw Thread

22, 22a: Sliding Stop Apparatus (Elevated Knot 22) Elevated Stop Ridge 22a)

### BEST MODE

Reference will now be made in greater detail to a preferred embodiment of the invention, an example of which is illustrated in the accompanying drawings. Wherever possible, the same reference numerals will be used throughout the drawings and the description to refer to the same or like parts.

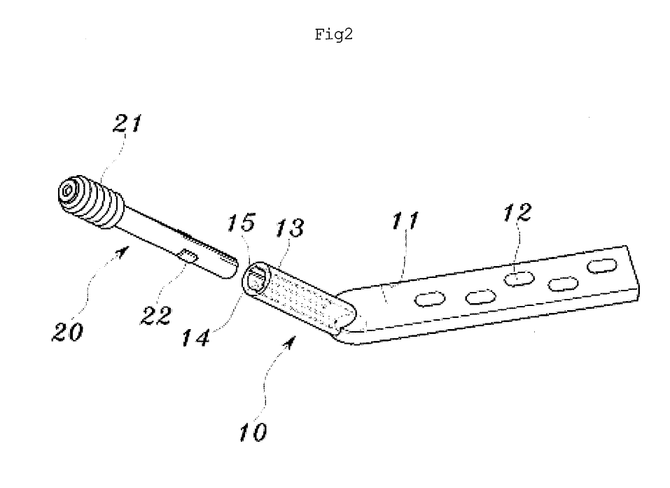

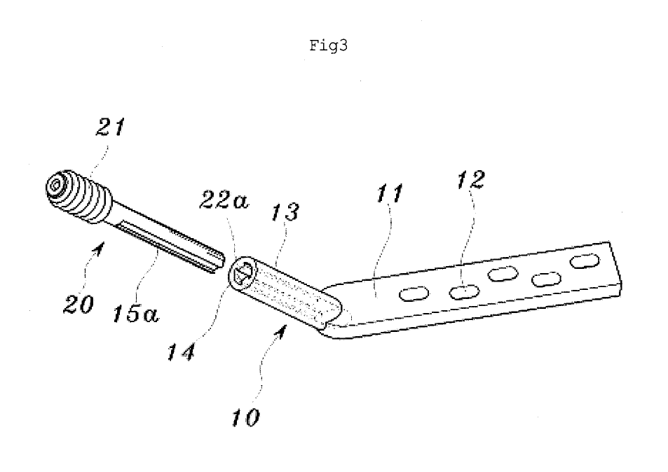

FIGS. 2 and 3 are perspective views illustrating the first and second embodiments of a device for preventing distal migration of a LAG screw used for hip joint fracture surgery in accordance with the present invention.

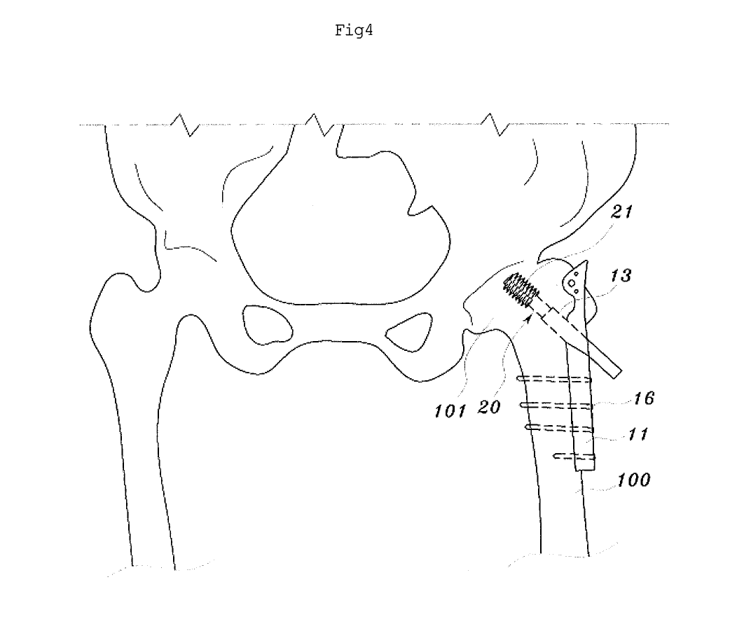

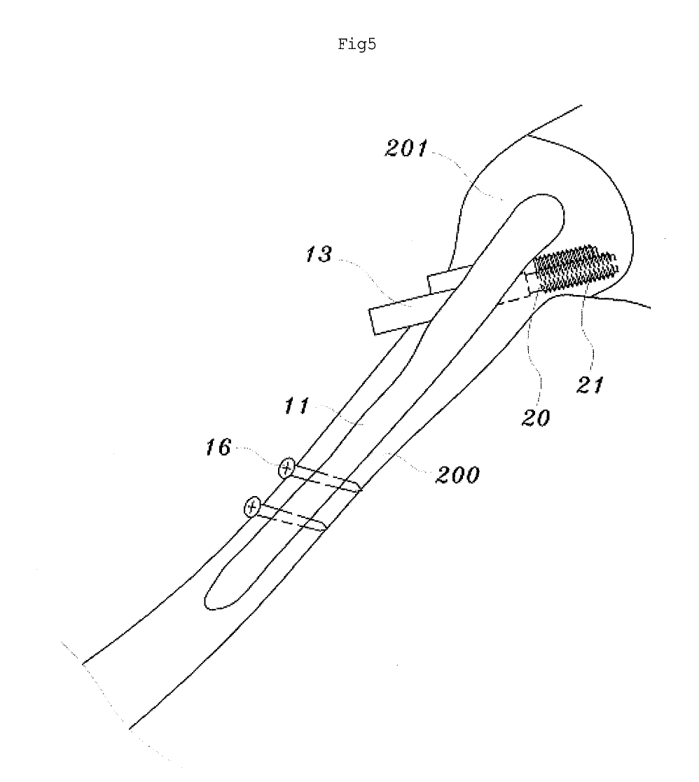

Referring to FIGS. 2 and 3, the device for preventing distal migration of a LAG screw used for hip joint fracture surgery in accordance with the present invention serves as a fracture repositioning device for surgery which is used to perform repositioning after a pertrochanteric fracture due to osteoporosis. The device comprises a Side Plate and Nail Rod 11 which is locked to a femur (100 in FIGS. 4 and 200 in FIG. 5) and a LAG screw which is locked to a Femoral head on top of the femur.

Describing the first embodiment of the device for preventing distal migration of a LAG screw used for hip joint fracture surgery in accordance with the present invention, the device includes a locking support 10 having a Side Plate or Nail Rod 11 on one part thereof and a LAG Screw Insertion Opening composed of LAG Screw Sliding Hole 14 having a the LAG Screw Insertion Opening 13 on the other part thereof, which are bent relative to each other. The Side Plate or Nail Rod 11 is locked to the femur 100. To this end, the Side Plate and Nail Rod 11 is defined with a plurality of Plate and nail screw hole 12 through which locking screws 16 (see FIG. 4) can be driven. The LAG Screw Insertion Opening 13 is composed by LAG Screw Sliding Hole 14 having the LAG Screw Insertion Opening 13. Depressed Stop Groove 15 is defined on one side or on each of opposite sides in the middle of the inner surface of the LAG Screw Sliding Hole 14. The angle between the Side Plate and Nail Rod 11 and the LAG Screw Insertion Opening 13 varies depending upon the configuration of the femur 100.

When the LAG screw is coupled to the LAG Screw Insertion Opening 13, the Shank of LAG screw 20 is inserted into the LAG Screw Sliding Hole 14 through the LAG Screw Insertion Opening 13. A Screw Thread 21 is formed on the proximal end of the LAG screw, and a Sliding Stop Apparatus (Elevated Knot) 22 is formed on one side or on each of opposite sides in the middle of the outer surface of the Shank of LAG screw 20 to be engaged in the Depressed Stop Groove 15 of the LAG Screw Sliding Hole 14 and to thereby prevent migration of the distal end of the LAG screw.

The length of the Depressed Stop Groove 15 defined in the LAG Screw Sliding Hole 14 and the position and size of the Sliding Stop Apparatus (Elevated Knot) 22 formed on the Shank of LAG screw 20 are appropriately determined in consideration of load applied to the bone migration prevention device for surgery attached to the bone and a patient's skeletal structure.

Describing the second embodiment of the device for preventing distal migration of a LAG screw used for hip joint fracture surgery in accordance with the present invention, a Screw Thread 21 is formed on the proximal end of the LAG screw, and the Sliding Stop Apparatus (Depressed Stop Groove) 15a is defined on one side or on each of opposite sides in the middle of the outer surface of the Shank of LAG screw 20.

The device includes a locking support 10 having a Side plate and Nail rod 11 on one part thereof and a LAG Screw Insertion Opening composed of LAG Screw Sliding Hole 14 having a the LAG Screw Insertion Opening 13 on the other part thereof, which are bent relative to each other. The Side plate and Nail rod The locking support 10 includes a Side Plate and Nail Rod 11 is locked to the femur 200. To this end, Side Plate or Nail Rod 11 is defined with a plurality of Plate and nail screw holes 12 through which locking screws 16 (see FIG. 5) can be driven. The LAG Screw Insertion Opening is composed by LAG Screw Sliding Hole 14 having the LAG Screw Insertion Opening 13. Sliding Stop Apparatus (Elevated Stop Ridge) 22a is projectedly formed on one side or on each of opposite sides in the middle of the inner surface of the LAG Screw Sliding Hole 14 to prevent migration of the distal end of LAG screw.

When the LAG screw is coupled to the LAG Screw Insertion Opening, the Shank of LAG screw 20 is inserted into the LAG Screw Sliding Hole 14 through the LAG Screw Insertion Opening 13, and the Sliding Stop Apparatus (Depressed Sliding Groove) 15a is engaged with the Sliding Stop Apparatus (Elevated Stop Ridge) 22a.

The position and size of the Sliding Stop Apparatus (Elevated Stop Ridge) 22a formed in the LAG Screw Sliding Hole 14 and the length of the Sliding Stop Apparatus (Depressed Sliding Groove) 15a defined on the Shank of LAG screw 20 are appropriately determined in consideration of the load applied to the migration prevention device attached to the bone and a patient's skeletal structure.

Of course, the present invention can be applied to surgical instruments such as a dynamic compression hip screw, a proximal femoral nailing, and so forth, which are conventionally used and adopt the principle as described above.

Hereafter, workings of the bone migration prevention device for surgery according to the present invention, constructed as mentioned above, will be described.

FIGS. 4 and 5 are schematic views illustrating the in-use state of the device for preventing distal migration of a LAG screw used for hip joint fracture surgery in accordance with the first and second embodiments of the present invention.

When installing the device for preventing distal migration of a LAG screw used for hip joint fracture surgery in accordance with the present invention, in the case of the first embodiment, the LAG screw is inserted into the LAG Screw Sliding Hole 14 such that the Sliding Stop Apparatus (Elevated Knot) 22 of the Shank of LAG screw 20 is engaged in the Depressed Stop Groove 15 of the LAG Screw Sliding Hole 14. In this state, the Screw Thread 21 of the LAG screw is driven into the femoral head. Then, with the Side Plate and Nail Rod 11 brought into close contact with the femur 100, and locked to the femur 100 by driving the locking screws 16 through the Plate and nail screw hole 12. In this way, the fractured femur 100 is repositioned.

In the case of the second embodiment, the LAG screw is inserted into the LAG Screw Sliding Hole 14 such that the Depressed Sliding Groove 15a of the Shank of LAG screw 20 is engaged with the Sliding Stop Apparatus (Elevated Stop Ridge) 22a of the LAG Screw Sliding Hole 14. In this state, the Screw Thread 21 of the LAG screw is driven into the Femoral head 201 with the Side Plate and Nail Rod 11 brought into close contact with the femur 200 by screw insertion 16, through the Plate and nail screw hole 12. In this way, the fractured femur 200 is repositioned.

As the device for preventing distal migration of a LAG screw used for hip joint fracture surgery in accordance with the present invention is locked to the femurs 100 and 200 as shown in FIGS. 4 and 5, the Sliding Stop Apparatus (Elevated Knot 22 and Elevated Stop Ridge 22a) are engaged into the Depressed Stop Groove 15 and the Depressed Sliding Groove 15a, as a result of which the Shank of LAG screw 20 is prevented from slidingly migration.

### INDUSTRIAL APPLICABILITY

As is apparent from the above descriptions, the device for preventing distal migration of a LAG screw used for hip joint fracture surgery according to the present invention provides advantages in that, since a migration stopper is formed on the Shank of LAG screw and in the LAG Screw Sliding Hole of the LAG Screw Insertion Opening, problems frequently occurring due to excessive migration of the distal end of the LAG screw after conducting surgery for repositioning a fractured part of a hip joint, such as malunion and nonunion of the fractured part, can be solved, whereby union of the fractured part is facilitated and development of complications is prevented.

Although preferred embodiments of the present invention have been described for illustrative purposes, those skilled in the art will appreciate that various modifications, additions and substitutions are possible, without departing from the scope and spirit of the invention as disclosed in the accompanying claim.

1. A device for preventing distal migration of a LAG screw used for hip joint fracture surgery, comprising:
a locking support having a Side Plate and Nail Rod on one part thereof and a LAG Screw Insertion Opening on the other part thereof, which are bent relative to each other, the Side Plate and Nail Rod having a plurality of Plate and nail screw holes through which the Side Plate and Nail Rod is locked to a bone, the LAG Screw Insertion Opening having a LAG Screw Sliding Hole, and a Depressed Stop Groove being defined on a side or on each of opposite sides in the middle of an inner surface of the LAG Screw Insertion Opening which defines the LAG Screw Sliding Hole; and a Shank of LAG screw inserted into the LAG Screw Sliding Hole of the LAG Screw Insertion Opening, and having a Screw Thread which is formed on a proximal end thereof and a Sliding Stop Apparatus (Elevated Knot) which is formed on a side or on each of opposite sides in a middle of an outer surface of the Shank of LAG screw to prevent migration of a distal end of the Shank of LAG screw.

2. A device for preventing distal migration of a LAG screw used for hip joint fracture surgery, comprising:
a Shank of LAG screw having a Screw Thread which is formed on a proximal end thereof and a Depressed Sliding Groove which is defined on a side or on each of opposite sides in a middle of an outer surface of the Shank of LAG screw; and
a locking support having a Side Plate and Nail Rod on one part thereof and a LAG Screw Insertion Opening on the other part thereof which are bent relative to each other, the Side Plate and Nail Rod having a plurality of Plate and nail screw holes through which the Side Plate and Nail Rod is locked to a bone, the LAG Screw Insertion Opening having a LAG Screw Sliding Hole, a Sliding Stop Apparatus (Elevated Stop Ridge) being projectedly formed on a side or on each of opposite sides in a middle of an inner surface of the LAG Screw Insertion Opening to prevent migration of a distal end of the Shank of LAG screw, and the Shank of LAG screw being inserted into the LAG Screw Insertion Opening such that the Depressed sliding Groove is engaged with the Sliding Stop Apparatus (Elevated Stop Ridge).

3. An apparatus comprising:
a locking support comprising a side plate and a LAG screw insertion opening; and
a LAG screw configured to slide into the LAG screw insertion opening, wherein the LAG screw and the LAG Screw insertion opening have substantially complementary shapes configured to prevent distal migration of the LAG screw from the LAG screw insertion opening.

4. The apparatus of claim 3, wherein the apparatus is device for preventing distal migration of a LAG screw during hip joint fracture surgery.

5. The apparatus of claim 3, wherein the side plate and the LAG screw insertion opening are formed at an angle.

6. The apparatus of claim 3, wherein the side plate comprises a plurality of holes through which the side plate is coupled to a bone.

7. The apparatus of claim 3, wherein said complementary shapes comprise:
the LAG screw comprising at least one groove; and
the LAG screw opening comprising at least one protruding feature, wherein the protruding feature is substantially complementary to the groove.

8. The apparatus of claim 7, wherein:
said at least one groove comprises one groove; and
said at least one protruding feature comprises one protruding feature.

9. The apparatus of claim 7, wherein:
said at least one groove comprises two grooves; and
said at least one protruding feature comprises two protruding features.

10. The apparatus of claim 7, wherein said at least one protruding feature is an elevated knot.

11. The apparatus of claim 7, wherein the shape of said at least one groove and said at least one protruding feature are configured based on a predetermined load applied to the apparatus.

12. The apparatus of claim 3, wherein said complementary shapes comprise:
the LAG screw comprising at least one ridge; and
the LAG screw opening comprising at least one groove, wherein the ridge is substantially complementary to the groove.

13. The apparatus of claim 12, wherein:
said at least one groove comprises one groove; and
said at least one ridge comprises one ridge.

14. The apparatus of claim 12, wherein:
said at least one groove comprises two grooves; and
said at least one ridge comprises two ridges.

15. The apparatus of claim 12, wherein the shape of said at least one groove and said at least one ridge are configured based on a predetermined load applied to the apparatus.
